(19) United States
(12) Patent Application Publication
(10) Pub. No.: US 2020/0121651 A1
(43) Pub. Date: Apr. 23, 2020

(54) Pramipexole for Use in the Treatment of Pain

(71) Applicant: STC.UNM, Albuquerque, MN (US)

(72) Inventors: Erin Damita Milligan, Placitas, MN (US); Patricio Rodrigo Escalona, Albuquerque, MN (US)

(21) Appl. No.: 16/656,171

(22) Filed: Oct. 17, 2019

Related U.S. Application Data
(60) Provisional application No. 62/747,447, filed on Oct. 18, 2018.

(51) Int. Cl. A61K 31/428; A61K 31/485; A61K 33/00; A61K 31/05; A61K 31/195; A61K 31/197; A61P 23/00

(52) U.S. Cl. CPC A61K 31/428; A61K 31/485; A61K 33/00; A61P 23/00; A61K 31/195; A61K 31/197; A61K 31/05

(57) ABSTRACT

The present invention is directed to the discovery that pramipexole is useful as an agent for the treatment of pathological pain, especially including chronic pain, including chronic nociceptive pain (principally from inflammation) and neuropathic pain, including peripheral neuropathy and central neuropathy. The action of pramipexole in treating pain is non-addictive. In addition, it has been discovered that pramipexole may be co-administered with an opioid agent in the treatment of pain in order to treat pain and reduce the development of tolerance of a patient or subject to the opioid agent such that the opioid agent may have a lasting effect without the requirement of escalating doses, compared to its administration in the absence of pramipexole. Additional embodiments of the present invention are described.

### RELATED APPLICATIONS

This application claims the benefit of priority of U.S. provisional application Ser. No. 62/747,447, of identical title, filed Oct. 18, 2018, the entire contents of which are incorporated by reference herein.

### GRANT SUPPORT

This invention was made with government support under R01-AA025967, R21-AA023051, T32-AA014127 and P50-AA022534 awarded by the National Institutes of Health (NIH) Alcohol Abuse and Alcoholism. The government has certain rights in the invention.

### FIELD OF THE INVENTION

The present invention is directed to the discovery that pramipexole is useful as an agent for the treatment of pain, especially including chronic pain, including chronic nociceptive pain (principally from inflammation) and neuropathic pain, including peripheral neuropathy and central neuropathy, as well as allodynia and hyperalgesia. The action of pramipexole in treating pain is non-additive. In addition, it has been discovered that pramipexole may be co-administered with an opioid agent in the treatment of pain in order to treat pain and reduce the development of tolerance of a patient or subject to the opioid agent such that the opioid agent may have a lasting effect compared to its administration in the absence of pramipexole.

### BACKGROUND AND OVERVIEW OF THE INVENTION

Opioid addiction has become a crisis in the United States. The rate of US opioid-related overdose deaths from 2013-2016 increased by an astonishing 88% per year, while overdose deaths due to other drugs remained virtually unchanged. These increasing opioid-related death rates are linked to increases in prescription opioid misuse (medical and non-medical) as well as illicit opioid use (heroin). Despite medical prescription opioid misuse generally declining during recent years, the number of individuals who misuse prescription and illicit opioids continues to increase. These data reveal an emerging opioid misuse epidemic (medical, non-medical, and illicit).

The public health crisis of prescription opioid and heroin abuse both regionally and nationally is unprecedented. While the underlying causes related to this public health crisis are largely speculative, one possible contributing factor is the dramatic escalation of prescription opioid treatment for chronic pain over the past decade. Preclinical studies convincingly support that not only do opioids exert negative consequences for pain, but also that chronic pain can be targeted by non-opioid drugs that lack direct actions on neurons thereby reducing addiction liability. Trafficking immune cells to the central nervous system (CNS) and immune-like glial cells within the CNS (e.g. astrocytes and microglia) are necessary for the development and maintenance of acute-to-chronic pain problems via the activation of the immune receptor, TLR4 causing the release of proinflammatory pronociceptive cytokines such as IL-1b.

Preclinical studies of the past few years show opioid drugs activate the same innate immune proinflammatory pathways such as TLR4 necessary for the production and release of IL-1b in both peripheral immune and glial cells. Non-opioid compounds that block the TLR4-IL-1b pathway may provide substantially better therapeutic benefit for opioids when these non-opioid compounds are co-delivered along with opioids, or may even exert superior pain relief when delivered alone. Therefore, the co-inventors of the present application identified a non-opioid drug that blocks TLR4 and also harnesses endogenous anti-inflammatory mechanisms resulting in the suppression of proinflammatory cytokines such as IL-1b, providing a novel approach to treat chronic pain in people while lacking potential for addictive side effects. Pramipexole is an FDA-approved drug used to treat Parkinson's Disease and Restless Legs Syndrome that demonstrates an excellent safety profile in humans while also demonstrating the above-noted anti-inflammatory profile in preclinical studies. The current data show pramipexole, a repurposed drug as a non-opioid pain therapeutic, inhibits the TLR4-IL-1b activation pathway in a cell-based assay (using human THP-1 cells), and completely abolishes allodynia in a mouse model of chronic peripheral neuropathy. Based upon the experimental results presented herein, the co-inventors predict that low doses of opioids will be sufficient to control pain when delivered along with pramipexole, and that tolerance to opioids is reduced and/or completely prevented, as pramipexole blocks the downstream effects of TLR4 activation. Additionally, pramipexole evidences activity consistent with its use to inhibit and/or eliminate withdrawal from opioid analgesics at low dose levels of opioid.

### BRIEF DESCRIPTION OF THE INVENTION

The present invention is directed to the use of pramipexole or its pharmaceutically acceptable salt in the treatment of a subject or patient for pain, especially chronic pain, including nociceptive pain and neuropathic pain (both peripheral neuropathic and central neuropathic pain), including allodynia and hyperalgesia. In embodiments, the subject or patient does not suffer from Parkinson's disease (PD) or restless legs syndrome (RLS). In an embodiment, pramipexole is the only agent administered to a patient or subject for the treatment of pain. In an embodiment, pramipexole is administered in combination with at least one other analgesic agent for the treatment of pain. In an embodiment, pramiprexole and at least one opioid analgesic agent are the only agents which are administered to a patient or subject for the treatment of pain. In further embodiments, pramipexole is administered in combination with at least one non-opioid analgesic agent for the treatment of pain.

The present invention is also directed to the use of pramipexole or its pharmaceutically acceptable salt in combination with an opioid analgesic agent in the treatment of pain, especially chronic pain, including nociceptive pain and neuropathic pain (both peripheral neuropathic and central neuropathic pain), including allodynia and hyperalgesia. Preferably, low doses of the opioid analgesic agent (at daily dosages ranging from about 0.01 mg to about 10 mg, more often 0.05 to about 10 mg, 0.1 mg to about 10 mg, 0.25 mg to about 5 mg, about 0.5 to 2.5 mg, about 0.75 to 1.75 mg, about 0.9 to 1.1 mg and about 1.0) are administered to a patient or subject in combination with pramipexole at dosages of pramipexole ranging from 0.05 to 5 mg, from 0.1 to 2.5 mg, from 0.25 to 2.0 mg, 0.5 to 1.5 mg, 0.75 to 1.25 mg and 0.9 to 1.1 mg (the ratio of the dose (weight) of opiate to pramipexole falling within a range of 200 to 0.002, often 50 to 0.02, more often 10 to 0.1, 5 to 0.5, 2.5 to 0.25, 2.0 to 0.75 and about 0.9 to 1.1, including about 1.0 to 1.0) which results in resolution of pain, and a reduction and/or elimination of tolerance, including a permanent elimination of tolerance by the patient or subject to the opioid analgesic agent.

In embodiments, co-administration of pramipexole with opioid analgesic agent may be used to reduce and/or eliminate withdrawal symptoms typically associated with the administration of opioid analgesic agents. In an embodiment, an effective amount of pramipexole is administered to a subject or patient in combination with an opioid analgesic agent such that withdrawal symptoms associated with opioid analgesic agent administration are reduced and/or eliminated. Thus, pursuant to this embodiment, a patient or subject who is being treated for pain is administered pramipexole in combination with an opioid analgesic agent, such that when administration of the combination of drugs is reduced and/or ceased, withdrawal symptoms are reduced and/or eliminated. In this embodiment of the present invention, the administration of an effective amount of pramipexole in combination with low doses of an opioid analgesic agent are used to reduce the withdrawal symptoms of the opioid analgesic agent. The dose of pramipexole which is used to effect a decrease in withdrawal in the subject or patient ranges from 0.05 to 5 mg, from 0.1 to 2.5 mg, from 0.25 to 2.0 mg, 0.5 to 1.5 mg, 0.75 to 1.25 mg and 0.9 to 1.1 mg in the presence of an opioid analgesic and the opioid analgesic is administered at a dosage ranging from about 0.01 mg to about 10 mg, more often 0.05 to about 10 mg, 0.1 mg to about 10 mg, 0.25 mg to about 5 mg, about 0.5 to 2.5 mg, about 0.75 to 1.75 mg, about 0.9 to 1.1 mg and about 1.0. The ratio of opiate to pramipexole which may be used to limit, reduce or eliminate withdrawal of the opiate falls within the dose ratio (weight) of opiate to pramipexole range of 200 to 0.002, often 50 to 0.02, more often 10 to 0.1, 5 to 0.5, 2.5 to 0.25, 2.0 to 0.75 and about 0.9 to 1.1, including about 1.0 to 1.0. In preferred embodiments, the amount of pramipexole is adjusted to a weight ratio which is at least 0.5 the dosage (weight) of opioid administered.

Opioid analgesic agents which may be combined with pramipexole in the treatment of pain include codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentanil and mixtures thereof. Non-opioid analgesics include gabapentin, pregabalin, cannabidiol oil (CBD oil), nitric oxide (e.g. noni, especially noni concentrate) and mixtures thereof.

In embodiments, the present invention is directed to a method for eliminating the tolerance of a patient or subject to opioid analgesic therapy, thus making the effect of the opioid greater, comprising administering to the subject or patient an effective amount of pramipexole in combination with an opioid analgesic agent where the effectiveness of the opioid analgesic agent in reducing and/or eliminating pain in the patient increases by at least half of day, often one day, often two or three days, four to seven days and up to several weeks or more (e.g. at least a month or two), more often several days to a week or more from the co-administration of pramipexole with at least one opioid analgesic agent. In embodiments, tolerance to the opioid analgesic agent lasts as long as the opioid analgesic agent is actually administered, such that the effect is indefinite.

In embodiments, the co-administration of pramipexole in combination with at least one opioid analgesic agent will reduce the likelihood of withdrawal and/or the symptomology associated with opioid analgesic withdrawal in a patient or subject being administered pramipexole and the opioid analgesic agent for the treatment of pain.

In embodiments, the present invention is also directed to a pharmaceutical composition comprising an anti-pain effective amount of pramipexole in combination with an effective amount of at least one opioid analgesic agent, the combination of agents being further combined with at least one pharmaceutically acceptable carrier, additive and/or excipient, wherein the opioid analgesic agent is selected from the group consisting of codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentail and mixtures thereof. In alternative embodiments, pramipexole is formulated with a non-opioid analgesic agent for the treatment of pain.

Other embodiments of the present invention may be readily gleaned from a review of the detailed description of the invention presented herein below.

### BRIEF DESCRIPTION OF THE FIGURES

FIG. 1 shows that intravenous injection of Pramipexole reduces hindpaw sensitivity male mice and produces complete reversal from allodynia, and is efficacious up to 2 hours. Absolute threshold behavioral responses for (A) ipsilateral and (B) contralateral hindpaw of mice subjected to either CCI or Sham surgery, in addition to an injection of Pramipexole or sterile saline (vehicle) at day 10 post surgery is shown above. At baseline behavioral hindpaw responses did not demonstrate any significant differences between mice of any group. Mice that were subjected to CCI surgery developed profound allodynia over a 10 day timecourse at which point the mice then received an intravenous injection of Pramipexole or sterile saline. Upon administration of Pramipexole, mice that received CCI surgery completely reversed from allodynia within 1 hour, with maximal efficacy seen at 2 hours post-injection. Mice that received Sham surgery remained at basal levels of sensitivity throughout the timecourse regardless of injection. Additionally, mice that were subjected to CCI and received a vehicle injection remained allodynic throughout the timecourse. These effects were identical from ipsilateral to contralateral hindpaws.

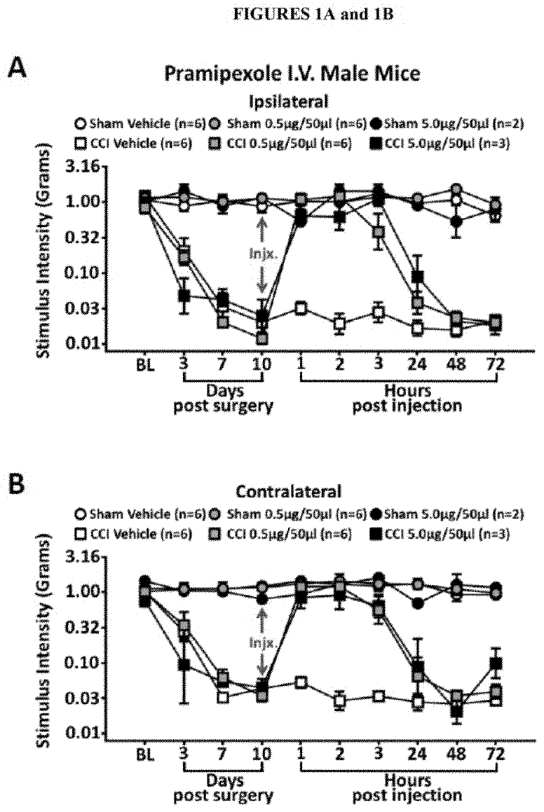

FIG. 2 shows that under cell culture conditions using a human monocyte cell line, THP-1 cells, pramipexole is capable of suppressing the pro-nociceptive cytokine, Interleukin-1beta (IL-1b), from being released after classic immune cell stimulation with the gram-negative bacteria cell wall particles, lipopolysaccharide (LPS). Pramipexole alone and two different doses (5 pg and 500 pg) does not significantly alter basal cellular levels of IL-1b. However, both 5 pg and 500 pg of pramipexole in the presence of LPS stimulation significantly blunts IL-1b release. These doses given to isolated cells in culture, 10,000 and 1000-fold lower than in in vivo doses, are significantly more vulnerable to drug effects due to the lack of typical in vivo counter-regulatory responses, such in degradative enzymes, that are typically activated from a plethora of surrounding cells.

### DETAILED DESCRIPTION OF THE INVENTION

The following terms shall be used throughout the specification to describe the present invention. A term, which is otherwise not defined, has the same meaning as one of ordinary skill within the context of the use of that term would assign to the term. Note that all terms are used in context to avoid overlap and redundancy where applicable.

The singular forms “a,” “an,” and “the,” include plural referents unless expressly and unequivocally limited to one referent. Thus, for example, reference to “an inhibitor” can include two or more different compounds. As used herein, the term “include” and its grammatical variants are intended to be non-limiting, such that recitation of items in a list is not to the exclusion of other like items that can be substituted or other items that can be added to the listed items.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In accordance with the present invention there may be employed conventional molecular biology, microbiology, recombinant DNA techniques, chemical synthetic methods and pharmacological practices within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Cells, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning”, among others.

The term “patient” or “subject” refers to an animal, preferably a mammal, and even more preferably a human, in need of treatment or therapy for pain, especially chronic pain to which compounds according to the present invention are administered in order to treat that condition.

The term “about” when used before a numerical designation, e.g., temperature, time, amount, concentration, and such other, including a range, indicates approximations which may vary by (+) or (−) 20%, 10%, 5% or 1%, or any subrange or subvalue there between. Preferably, the term “about” when used with regard to a dose amount means that the dose may vary by +/−20%. For example, “about 2 mg/kg pramipexole” indicates that a patient may be administered a dose of pramipexole between 1.6 mg/kg and 2.4 mg/kg. In another example, about 1.0 mg per unit dose of premipexole indicates that the unit dose may range from 0.8 mg to 1.2 mg.

“Administration” refers to introducing an agent, such as pramipexole or an opiate, into a patient. Typically, an effective amount is administered, which amount can be determined by the treating physician or the like. Dosages for opiates and pramipexole which are used in the present invention are presented herein below. Any route of administration, such as oral, topical, subcutaneous, peritoneal, intra-arterial, intrathecal, inhalation, vaginal, rectal, nasal, introduction into the cerebrospinal fluid, or instillation into body compartments can be used. The agent, such as pramipexole or the opiate analgesic, may be administered by direct blood stream delivery, e.g. intravenous, sublingual, buccal, intranasal, or intrapulmonary administration.

The term “coadministration” shall mean that at least two compounds or compositions (i.e. pramipexole and at least one opioid analgesic agent) are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds according to the present invention may be administered with one or more additional bioactive agents, especially including an additional antibiotic for purposes of treating bacterial, especially gram negative bacteria.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of an agent, such as, pramipexole or its salt one, two, three, or more times per day. Administration may be via transdermal patch, gum, lozenge, sublingual tablet, intranasal, intrapulmonary, oral administration, intramuscularly or other administration.

The term “comprising” or “comprises” is used to indicate that the compositions and methods include the recited elements, but do not exclude other elements not recited. The term “consisting essentially of” when used to define compositions and methods, shall mean excluding other elements of any essential significance to the combination claimed for the stated purpose. Thus, a composition consisting essentially of the elements as defined herein would not exclude other materials or steps that do not materially affect the basic and novel characteristic(s) of the claimed invention. The term “consisting of” shall mean excluding more than trace elements of other ingredients and substantial method steps. Embodiments defined by each of these transition terms are within the scope of this invention.

The term “effective” is used herein, unless otherwise indicated, to describe an amount of a compound or component which, when used within the context of its use, produces or effects an intended result, whether that result relates to the prophylaxis and/or therapy of an infection and/or disease state, especially a bacterial infection including a infection within the context of its use or as otherwise described herein. The term effective subsumes all other effective amount or effective concentration terms (including the term “therapeutically effective”) which are otherwise described or used in the present application.

The term “therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of pramipexole, in the context of treating drug withdrawal, refers to an amount of pramipexole that attenuates the dependency and/or symptoms of acute withdrawal for at least 2 hours beyond control (placebo), at least 5 hours beyond control, and preferably at least 10 hours beyond control, for example at least 24 hours or more beyond control. Within the context of decreasing opioid tolerance, therapeutically effective amount refers to an amount of that decreases the tolerance to opioid analgesic therapeutic activity by at least two hours beyond control (placebo), at least 5 hours beyond control, at least 12 hours beyond control and often 24 hours or longer beyond control, as otherwise described herein. In certain embodiments, opioid tolerance is eliminated and the opioid may be administered indefinitely without the patient or subject exhibiting tolerance to the opioid.

The term “effective” is used to describe an amount of a compound or component which is included in a composition or in a method of treatment to effect its intended result, whether that result is the treatment of pain and/or the ability of the patient to exhibit increased tolerance to the administration of a an opioid analgesic agent.

The term “compound”, as used herein, unless otherwise indicated, refers to any specific chemical compound disclosed herein regardless of the treatment or the mechanism by which such effect occurs and includes in context, tautomers, regioisomers (especially cis/trans), geometric isomers, and where applicable, optical isomers thereof, as well as pharmaceutically acceptable salts, solvates and polymorphs thereof. Within its use in context, the term compound generally refers to a single compound (especially with respect to pramipexole and its pharmaceutically acceptable salts, especially its dihydrochloride salt, but also with respect to opioid and other analgesics and their pharmaceutically acceptable salts), but may also include other compounds such as stereoisomers, regioisomers and/or optical isomers (including in some instances, racemic mixtures) as well as specific enantiomers or enantiomerically enriched mixtures of disclosed compounds, depending on the context of the use of the term. The compounds of this invention include all stereoisomers where relevant (e.g., cis and trans isomers) and all optical isomers of the present compounds (eg., R and S enantiomers), as well as racemic, diastereomeric and/or other mixtures of such isomers, as well as all pharmaceutically acceptable salt forms, solvates, polymorphs and prodrug forms of the present compounds, where applicable.

The term “treat”, “treating” or “treatment” is used to describe the inhibition, amelioration and/or resolution of pain in a subject or patient pursuant to the administration of compounds or compositions according to the present invention. These terms are therapeutic terms. In the case of prophylaxis, prevention and/or reduction in the likelihood of pain occurring in a patient or subject can also be effected.

The term “pramipexole” is used to describe the compound (6S)-6-N-propyl-4,5,6,7-tetrahydro-1,3-benzothiazole-2,6-diamine (IUPAC) and its various pharmaceutically acceptable salts, including its dihydrogenchloride salt. Pramipexole, as its neutral chemical species has the following chemical structure:

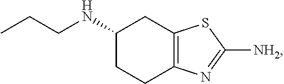

which also is available as the dihydrochloride salt. Pramipexole is a non-ergoline dopamine agonist which has been used traditionally to treat Parkinson's disease and restless leg syndrome.

The term “opioid analgesic” is used to describe any analgesic which exhibits opioid agonist activity. Opioids are substances that act on opioid receptors to produce morphine-like analgesic effects. Medically these agents are primarily used for pain relief, including anesthesia. Exemplary opioid analgesics for use in the present invention include the following agents: codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentanil and mixtures thereof.

The term “non-opioid analgesic” refers to an analgesic agent which provides pain relief and is not an opioid and is generally non-addictive. Examples of non-opioid analgesics include gabapentin, pregabalin, cannabidiol oil (CBD oil) and nitric oxide (e.g. noni, especially noni concentrate).

The term “pain” is used to describe an unpleasant feeling that is conveyed to the brain by sensory neurons. The discomfort signals actual or potential injury to the body. Pain is a sensation, the physical awareness of pain and it also includes perception, the subjective interpretation of the discomfort. Perception gives information on the pain's location, intensity, and something about its nature. The various conscious and unconscious responses to both sensation and perception, including the emotional response, add further definition to the overall concept of pain. The present invention may be used to treat pain, especially acute pain lasting more than several days and chronic pain as otherwise described herein. The treatment of pain using opioid analgesics in combination with pramipexole occurs consistent with the use of opioid analgesics in the treatment of pain. In most instances, pramipexole is used in the treatment of chronic pain as described herein.

The term “acute pain” is used to describe pain, often of short duration, but which may last for several weeks or more which is provoked by a specific disease or injury, serves a useful biologic purpose, is associated with skeletal muscle spasm and sympathetic nervous system activation, and is self-limited. The therapy of acute pain is aimed at treating the underlying cause and interrupting the nociceptive signals. The present invention is useful in the treatment of acute pain which tends to last more than a few days to a week or more.

The term “chronic pain” is used to describe pain which occurs on a lasting basis for at least about 2-3 months and includes nociceptive pain and neuropathic pain (both peripheral and central), including allodynia. Chronic pain, in contrast to acute pain, may be considered a disease state. Chronic pain is pain that outlasts the normal time of healing, if associated with a disease or injury. Chronic pain may arise from psychological states, often serves no biological purpose, and often has no recognizable end-point. The present invention is particularly effective in the treatment of chronic pain using pramipexole as a non-additive analgesic agent alone or in combination with an opioid analgesic agent as otherwise described herein. It is an unexpected that the co-administration of pramipexole in combination with an opioid analgesic analgesic agent lowers the tolerance for the opioid analgesic agent (i.e., reduces the tolerance of the patient or subject for the opioid such that the opioid will function as an analgesic agent at lower doses and for longer periods of time) when combined with pramipexole administration.

The term “allodynia” is used to describe a type of pain, generally on the skin, that's caused by something that wouldn't normally cause pain. This pain type is frequently associated with fibromyalgia, and some people with chronic fatigue syndrome have it as well. Other conditions associated with allodynia include neuropathy, shingles (posthermic neuralgia) and migraines. Allodynia comes in three different and distinct forms. It is possible for a subject to have one or more of these kinds of allodynia. The three types of allodynia are tactile allodynia, mechanical allodynia and thermal allodynia. Tactile allodynia is pain which is caused by touch. This can be caused by clothing lying against the skin (e.g., waistbands, straps, elastic bands on socks), a light touch on the skin or even a hug. Mechanical allodynia is pain which is caused by movement across the skin. This movement can be for example, a towel, bedsheets, clothing which rubs against the skin or even the air from a fan blowing over the skin. Thermal allodynia is pain which is caused by heat or cold that is not extreme enough to cause damage to tissue. For example, the skin of a subject may burn if chilled, or getting the skin too hot may make the skin ache. Although allodynia is principally associated with neuropathy in the present invention, allodynia associated with other causes are also treated by the present invention.

The term “coadministration” or “combination therapy” is used to describe a therapy in which at least two active compounds in effective amounts are used to treat a disease state or condition as otherwise described herein at the same time. Although the term coadministration preferably includes the administration of two active compounds to the patient at the same time, it is not necessary that the compounds be administered to the patient at the same time, although effective amounts of the individual compounds will be present in the patient at the same time. Pramipexole (which term includes any one or more of pramipexole or its pharmaceutically acceptable salts, solvates or polymorphs) according to the present invention may be administered with one or more opioid agonists which are traditionally used in the treatment of pain, especially chronic pain. These opioid agents include, for example, codeine, fentanyl, hydrocodone, hydromorphone, morphine, meperidine, oxycodone (alone or in combination with naloxone), among others as described herein. Coadministration of pramipexole and at least one additional opioid analgesic agent is effective to substantially reduce the tolerance of the patient to the administration of the opioid analgesic agent such that the opioid analgesic agent will exhibit far longer lasting effect on pain treatment than if the opioid agent was administered in the absence of pramipexole. In embodiments, tolerance to the opioid is eliminated.

The term “tolerance” is used to describe the effect that occurs over time from the treatment of pain using opioid analgesic agents wherein the patient or subject who is being treated with an opioid analgesic agent requires higher doses of the analgesic agent over time to produce the same effect in diminishing pain. In the present invention, the administration of pramipexole in combination with an opioid analgesic agent reduces the tolerance that the patient exhibits toward the opioid analgesic agent and in certain instances, actually eliminates tolerance.

The term “withdrawal” is used to describe symptoms which occur when an opioid analgesic agent is reduced in dosage or withdrawn from a subject or patient after a certain period of time (pain treatment). These symptoms are brief, severe and measurable and include, for example, rhinorrhea (excessive discharge), lacrimation (flow of tears), chills, gooseflesh (piloerection), hyperventilation, hyperthermia, mydriasis (pupil dilation), muscular aches, vomiting, diarrhea, anxiety & hostility, among others. Onset of withdrawal (also referred to as abstinence syndrome) depends on the opioid used. For example, morphine or heroin induced sudden abstinence onset could be 10 hrs after the last dose—peaks at 36-48 hrs and resolves by day 5. Others can persist for many months.

In embodiments, the co-administration of pramipexole and an opioid analgesic agent will reduce and/or eliminate at least one withdrawal symptom and preferably at least 2, 3, 4, 5, 6, 7, 8, 9, 10, 11 and 12 symptoms identified above as measured against a control (the symptoms are preferably reduced at least 50% compared to control often 60%, 65%, 75%, 85%, 90%, 95%, 99%, and in many instances withdrawal does not occur). Thus, coadministration of pramipexole and at least one additional opioid analgesic agent in combination is effective to substantially reduce the withdrawal of the patient to the administration of the opioid analgesic agent such that the opioid analgesic agent will exhibit attenuated withdrawal symptoms and in certain cases, the elimination of withdrawal symptoms upon reduction and/or cessation of pain therapy. In embodiments, effective amounts of pramipexole, alone or in combination with an opioid analgesic agent to a patient exhibiting withdrawal symptoms from opiates or anticipating withdrawal symptoms from the cessation of opiate therapy may attenuate or even eliminate the withdrawal symptoms from the cessation of the opiate therapy.

Thus, the present invention is directed to a method for treating pain, especially chronic pain, including nociceptive pain and neuropathic pain (both peripheral and central), including allodynia, comprising administering to a patient or subject in need an effective amount of pramipexole as the sole analgesic agent, or in combination with at least opioid analgesic agent, including an opioid analgesic agent selected from the group consisting of codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentanil and mixtures thereof. It has been discovered that the coadministration of pramipexole in combination with at least one opioid analgesic agent will decrease the tolerance of the patient or subject being administered the opioid analgesic agent to the opioid agent and to limit or minimize the dosage of opioid which can be used to treat pain. The term “tolerate” is used to describe the use of opioid analgesics which occurs over a period of time in a subject or patient such that in order to exhibit the same level of pain therapy in the subject or patient, the subject or patient must be administered an increasingly larger dose over time. Quite unexpectedly, the use of pramipexole in combination with an opioid analgesic agent reduces the tolerance that the patient or subject exhibits to the opioid analgesic in the absence of pramipexole, often substantially lowering the dose of opioid analgesic that can be administered to treat pain.

The present invention is also directed to pharmaceutical compositions which consist essentially of an effective amount of pramipexole in combination with an effective amount of at least one additional analgesic compound which is an opioid analgesic agent or a non-opioid analgesic agent, preferably an opioid analgesic agent selected from the group consisting of codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentanil and mixtures thereof, optionally in combination with an effective amount of a pharmaceutically acceptable carrier, additive or excipient, where the pramipexole is included in said composition preferably in an amount effective to reduce the tolerance of the patient to the administration of the opioid analgesic agent such that the amount of opioid analgesic agent can be reduced and/or the duration of administration of the opioid analgesic agent can be extended to effect pain relief. In alternative embodiments, the analgesic agent is a non-opioid analgesic agent selected from the group consisting of gabapentin, pregabalin, cannabidiol oil (CBD oil) and nitric oxide (e.g. noni, especially noni concentrate), among others.

The present invention also includes the compositions comprising the pharmaceutically acceptable salt. i.e., the acid or base addition salts of compounds of the present invention and their derivatives. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others. In a preferred aspect the dihydrochloride salt of pramiprexole (pramiprexole dihydrochloride or Mirapex) is used as the active pramiprexole agent.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines, among others.

Regardless of the mechanism, the compounds of the present invention may be used to treat disease states or conditions in patients or subjects who suffer from those conditions or disease states or are at risk for those conditions. In this method a compound in an effective amount is administered to a patient in need of therapy to treat pain and associated conditions.

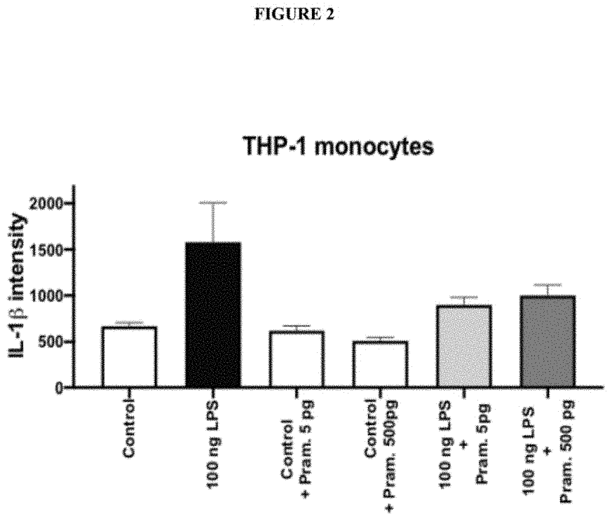

Without being limited by way of theory, it is believed that pramipexole is involved in the inhibition of the TLR4-IL-1b activation pathway to provide its unexpectedly effective result in the present invention, as demonstrated in FIG. 2. Preclinical studies of the past few years show opioid drugs activate the same innate immune proinflammatory pathways such as TLR4 necessary for the production and release of IL-1b in both peripheral immune and glial cells. Non-opioid compounds that block the TLR4-IL-1b pathway may provide substantially better therapeutic benefit for opioids when these non-opioid compounds are co-delivered along with opioids, or may even exert superior pain relief when delivered alone. The current data unexpectedly show pramipexole as a non-opioid pain therapeutic, which inhibits the TLR4-IL-1b activation pathway in a cell-based assay (using human THP-1 cells), and completely abolishes allodynia in a mouse model of chronic peripheral neuropathy. Based upon the experimental results presented herein, the co-inventors believe that low doses of opioids are especially effective to control pain when delivered along with pramipexole, and that tolerance to opioids is reduced and/or completely prevented, as pramipexole blocks the downstream effects of TLR4 activation. Additionally, pramipexole evidences activity consistent with its use to inhibit and/or eliminate withdrawal from opioid analgesics alone or in combination with low dose levels of opioid as otherwise described herein.

Compositions according to the present invention may be administered by any conventional means known in the art. Pharmaceutical formulations include those suitable for oral, rectal, nasal, topical (including buccal and sub-lingual), vaginal or parenteral (including intramuscular, sub-cutaneous, intrathecal and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above-described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier, additive or excipient. In general, it is preferable to administer the pharmaceutical composition orally, but certain formulations may be preferably administered parenterally and in particular, in intravenous, intrathecal or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous, intrathecal and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration, where applicable, can be prepared by mixing an active agent and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the active.

Dosage forms for topical administration include ointments, powders, sprays and inhalants. The compound(s) are admixed under sterile conditions with a physiologically acceptable carrier, and any preservatives, buffers, and/or propellants that may be required. Opthalmic formulations, eye ointments, powders, and solutions are also contemplated as being within the scope of this invention.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and the type of pain to be treated, and the particular mode of administration. In embodiments, pramipexole and opioid analgesic agent are used in effective amounts. Preferably, the compositions should be formulated to contain between about 0.005 milligram to about 5 milligrams or more, more preferably about 0.01 milligram to about 5 milligrams, and even more preferably about 0.1 milligrams to about 2.5 mg of pramipexole and optionally, at least 0.01 milligrams to about 10 milligrams or more, about 0.05 mg to about 5 milligrams and about 0.1 mg to about 2. 5mg of at least one opioid analgesic agent as otherwise described herein. Often, low doses of the opioid analgesic agent (at dosages ranging from about 0.01 mg to about 10 mg, more often 0.05 to about 10 mg, 0.1 mg to about 10 mg, 0.25 mg to about 5 mg, about 0.5 to 2.5 mg, about 0.75 to 1.75 mg, about 0.9 to 1.1 mg and about 1.0) are administered to a patient or subject in combination with pramipexole at dosages of pramipexole ranging from 0.05 to 5 mg, from 0.1 to 2.5 mg, from 0.25 to 2.0 mg, 0.5 to 1.5 mg, 0.75 to 1.25 mg and 0.9 to 1.1 mg (the ratio of the dose (weight) of opiate to pramipexole falling within a range of 200 to 0.002, often 50 to 0.02, more often 10 to 0.1, 5 to 0.5, 2.5 to 0.25, 2.0 to 0.75 and about 0.9 to 1.1, including about 1.0 to 1.0) which results in resolution of pain, and a reduction and/or elimination of tolerance, including a permanent elimination of tolerance by the patient or subject to the opioid analgesic agent.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular pain condition being treated.

### EXAMPLES

Materials and Methods:

Animals

The current report analyzed adult (10-16 week old) male C57BL/6 mice (wildtype; FFID: IMSR_JAX:000664) purchased from Jackson Laboratories (Bar Harbor, Me., USA). Mice were maintained on a standard 12:12 light/dark cycle (lights on from 0600 hours to 1800 hours) in a temperature controlled environment (23°±2° C.) for the duration of the study. Additionally, mice were housed in groups of 4-5 mice/cage, of which all mice were routinely monitored by the animal care staff under the direction of the institutional veterinarian, fed standard rodent chow and water ad libitum, with cages and bedding changed every 7 days. All procedures were approved by the Institutional Animal Care and Use Committee (IACUC) of the University of New Mexico Health Sciences Center, conducted in accordance to the NIH Guidelines for the Care and Use of Laboratory Animals, and closely adhered to guidelines from the International Association for the Study of Pain for the use of animals in research (Foundation for Biomedical Research, The Biomedical Investigator's Handbook for Researchers Using Animal Models. Washington, D.C.: FBR, 1987. WWW: http://www.fbresearch.org/).

Chronic Constriction Injury (CCI)

This study utilized a modified version of a well-established rat peripheral nerve injury model in which pathological sensitivity to light touch, clinically termed allodynia, is induced through a chronic constriction injury (CCI) (Bennett and Xie 1988). Introducing minor modifications to the Bennett and Xie rat model of neuropathic pain has demonstrated the ability to replicate this model of neuropathic pain in mice (Liu et al. 2017; Martucci et al. 2008; Vanderwall et al. 2018). Under isoflurane anesthesia (1.5-2.0% volume in oxygen, 2.0 l/min), the dorsal left thigh was shaved and cleaned using 70% Ethanol (EtOH) that was air dried prior to surgery. Utilizing a small incision over the posterior thigh, remaining 3 cm below the femur bone, from hip bone to the patella, the muscle fascia of the mouse was exposed. Using aseptic procedures, a blunt dissection was performed to carefully isolate the sciatic nerve by separating the overlying muscular fascia. Sterile plastic probes were then used to locate and lift the sciatic nerve from its pocket within the muscle tissue. Mice were then subjected to unilateral loose ligation of the sciatic nerve with three 5-0 chromic gut sutures (Ethicon; Somerville, N.J., Cat #634G) which were snugly tied around the sciatic nerve (avoiding pinching) proximal to the trifurcation with 1.5 mm spacing between sutures. Throughout the duration of the surgery, sterile 0.9% isotonic saline (Hospira; Cat #NDC 0409-4888-03) was irrigated over the sciatic nerve to prevent dehydration. Sham surgery followed the same CCI surgical procedure identically but without ligation. Upon ligation, the sciatic nerve was gently placed back into its correct anatomical position using the sterile plastic probes, and the overlying muscle fascia was stitched closed using one 4-0 sterile silk sutures (Ethicon; Somerville, N.J., Cat #83G). Wound clips (Kent Scientific Corp.; Cat #INS750344) were then utilized to seal the incision closed at which point the surgical procedure was complete. Mice fully recovered from anesthesia within approximately 10 minutes and were monitored daily for post-operative complications such as infection and/or irritation, additionally wound clips were removed approximately 5 days post-surgery. Body weight was monitored prior to and after surgery. All mice in this study recovered completely from the surgery, without any abnormalities, and remained subjects in the study.

Behavioral Assessment of Allodynia

Upon a 7-14 day acclimation to the testing environment, mice were subjected to a habituation period. The habituation period included placing the mice atop a testing rack within the first 4 hours of the light cycle (lights on from 0600 hours to 1800 hours) in a sound and temperature (23°±2° C.) controlled environment, allowing full access to the plantar hindpaw. Mice were exposed to four separate habitation periods for approximately 45 min/day. Sensitivity to mechanical light touch, clinically termed allodynia, was assessed using the von Frey behavioral assay in which calibrated monofilaments were applied to the hindpaw to elicit a paw withdraw response. Hindpaw threshold responses to light mechanical stimuli were assessed by adopting principles of the von Frey fiber test originally developed for the rat (Bonin, Bories, and De Koninck 2014; Chaplan et al. 1994; Milligan et al. 2000; Sommer and Schafers 1998), and modified for the mouse, as described in below briefly (Vanderwall et al. 2018). The von Frey behavioral assay was applied using nine calibrated monofilaments (touch-test sensory evaluator: North Coast Medical; Cat #NC12775) applied to the plantar surface of both the left and right hindpaws at random for a maximum of 3.0 s, with repeated stimulus presentations to a single animal using a minimum inter-trial stimulus period of 30 s. The log intensity of the nine monofilaments used is defined as log₁₀ (grams×10,000) with the range of intensity being as follows reported in log (grams): 2.36 (0.022 g), 2.44 (0.028 g), 2.83 (0.068 g), 3.22 (0.166 g), 3.61 (0.407 g), 3.84 (0.692 g), 4.08 (1.202 g), 4.17 (1.479 g), and 4.31 (2.042 g). Testing began at fiber marked 3.22 with subsequent monofilaments used based on the response/non-response of the mouse to the previous monofilament tested. If no response was elicited by the stimulus presented from the 3.22 monofilament, the next “heavier” monofilament was tested (e.g. 3.61). If a response was elicited by the stimulus presented from the 3.22 monofilament, the next “lighter” monofilament was tested (e.g. 2.83). A maximum total of six stimulus presentations were applied to each paw. The total number of positive and negative responses were then entered into the computer software program, PsychoFit (http://psych.colorado.edu/˜lharvey: RRID: SCR_015381) to determine the absolute withdrawal threshold (50% paw withdrawal threshold), as was previously described (Dengler et al. 2013; Milligan et al. 2000; Noor et al. 2017; Sanchez et al. 2017; Vanderwall et al. 2018). The PsychoFit program fits a Gaussian integral psychometric function to the observed withdrawal rates for each monofilament using a maximum-likelihood fitting method (Milligan et al. 2000). The behavioral responses were used to calculate the 50% paw-withdraw threshold (absolute threshold) which is then expressed as absolute log₁₀ stimulus intensity in (mg×10), or stimulus intensity (grams) on a linear scale. Data was then plotted using GraphPad Prism. Hindpaw assessment of mice occurred in groups of 4-6 per testing trial, additionally, behavioral assessment was conducted at baseline (BL), days post-surgery and hours post-injection as indicated graphically. Time points were choses to tease apart minimal differences between groups at key times while avoiding overstimulation.

Pramipexole Preparation

Pramipexole dihydrochloride, (S)-2-amino-4,5,6,7-tetrahydro-6-(propylamino) benzothiazole dihydrochloride (Sigma-Aldrich; CAS#:104632-25-9), was initially reconstituted in sterile 0.9% isotonic saline (Hospira; Cat #NDC 0409-4888-03) as a stock solution of 1 μg/μl, followed by creating 50, 100 μl aliquots (at a concentration of 100 μg in 100 μl saline) in a 0.5 ml sterile Eppendorf tube, which was then sealed with parafilm and stored at −80° C. for later use. On the day of injection, one aliquot was removed from −80° C. and was allowed to thaw at room temperature for 15 minutes. Upon reaching room temperature the stock aliquot of pramipexole was diluted using sterile 0.9% isotonic saline such that each 50 μl i.v. injection contained 5 μg or 0.5 μg pramipexole, which was then vortexed for approximately 15 s. Additionally, administration of vehicle followed the same procedure, however the 50 μl injection consisted solely of sterile 0.9% isotonic saline. Animals were injected within the hour following dilution.

Intravenous Injection (i.v.)

The current report aimed to analyze the development, magnitude and duration of allodynia upon an intravenous (i.v.) injection of the selective D2/D3 dopamine receptor agonist, pramipexole. For all experiments characterizing pramipexole efficacy, i.v. injection of pramipexole or vehicle into mouse tail veins occurred Day 10 post-surgery within 2.5 hours of the initiation of the light cycle. Using aseptic procedures, 50 μl of pramipexole or vehicle was collected into individual 1 cc, 27G ⅝ insulin syringes (Becton Dickinson; Cat #329412). Weights of the mice were recorded prior to injection, followed by placing mouse tails under a heat lamp to dilate the lateral tail vein, providing easier access to the vein. To safely heat the mouse tail without excessively heating the body, the tail was held firmly in place while a soft cloth was placed over the mouse body, leaving the tail exposed and the body shielded from heat. The mouse was then immediately moved to a plastic restraint which contained a slit through the middle, in which the tail is carefully moved through, allowing isolation and proper positioning of the tail for injection. With the tail held firmly in place the needle was then inserted into the lateral tail vein, followed by a small amount of blood efflux into the syringe with a subsequent 10 s injection. Following injection, sterile gauze was placed over the point of puncture utilizing a small amount of pressure to prevent excess bleeding. The mouse was then placed back in its home cage and for the following 5-10 minutes was monitored for any abnormal behavior or adverse side effects of injection. The total time required for handing and injection was approximately 2-3 minutes, with no need for anesthetics.

Statistical Analysis

All statistics were produced using IBM SPSS Statistics version 24 (IBM; RRID:SCR_002865). The assumption of sphericity was assessed using Mauchly's Test of Sphericity (α=0.05) and if the assumption of sphericity was violated (p>0.05), the reported degrees of freedom and p-values were adjusted using the applicable Greenhouse-Geisser or Huynh-Feldt correction to protect against Type-I errors. This experiment examined hind paw sensitivity in male mice following manipulation of the sciatic nerve though chronic constriction injury during a 13 day timecourse. Therefore, a 2 way repeated measures analysis of variance (ANOVA) was conducted in accordance to various timepoints throughout the experiment. Hindpaw sensitivity thresholds were assessed at BL, days 3,7 & 10 post surgery, as well as hours 1,2,3,24,48 & 72 post injection with a single corresponding ANOVA performed at BL with additional 2 way repeated measures ANOVA's performed at hours 1-3, hours 24-72 and hours 1-72 post injection. All hindpaw sensitivity data was graphed in GraphPad Prism version 7.02 (GraphPad Software Inc.; RRID:SCR_002798) and presented as the mean±Standard Error of the Mean (SEM).

THP-1 Cell Culture and Differentiation

THP-1 human acute monocytic leukemia cells were obtained from American Type Culture Collection (Manassas, Va., USA). THP-1 cells were grown in complete RPMI (ATCC modified RPMI supplemented with 10% fetal calf serum and 1% antibiotics). Cells were maintained at 0.5-1×10⁶ cells/ml in a humidified chamber at 37° C., in a mixture of 95% air and 5% CO2. RPMI/ATCC modified media was purchased from ThermoFisher Scientific, MA, USA (catalog #A1049101). Fetal calf serum (#97068-085) and Pen/Strep antibiotics (#450000-652) were purchased from VWR, PA, USA.

On the day of the experiment, cells were collected by centrifugation and suspended at 2.5×10⁶ cells/ml; 20 μl of cells were plated into individual wells of a 384 well tissue culture plate (Greiner, catalog #781280). Stock solution of LPS (Millipore Sigma, catalog #L2630, 5 mg/ml) was diluted in complete medium to prepare a 200 ng/ml solution (2× solution).

Control (complete media only) or LPS was added to respective wells in 25 μl volumes. Pramipexole stock solution (1 μg/μl) was thawed on ice and diluted to 10× concentrations (5 ng/ml or 50 pg/ml). 5 μl of pramipexole or complete medium was added to respective wells. The plate was placed on a plate shaker for 60 seconds for mixing. Cells were then incubated overnight (20hrs).

For phorbol myristate acetate (PMA)-differentiated experiments, THP-1 monocytes were differentiated to macrophages with PMA (Millipore-Sigma #524400), 40 nM) for 3 days. Wells were washed with fresh medium and the adherent cells were further cultured for 48 hours in complete media. Cells were then stimulated with LPS for 4 hours followed by an additional 20 hours with pramipexole. LPS (100 ng/ul) and pramipexole (5 and 500 pg/ml) concentrations were the same for both THP-1 monocytes and THP-1 macrophage stimulation experiments. Each culture condition was tested in four replicates.

IL-1β Detection

Culture supernatants were assayed for the presence of IL-1β using the MultiCyt® QBeads® Human PlexScreen Secreted Protein Assay Kit (Intellicyte, NM, USA). The assay was performed according to the manufacturer's instructions. Using a similar principle as sandwich ELISA, QBeads® technology allowed faster quantitative measurement of IL-1β levels from fresh supernatant samples. Briefly, following LPS and pramipexole treatments, supernatants were harvested from individual wells and placed in 384 well assay plates (Eppendorf twintec) and analyzed immediately. Samples were added to wells containing the IL-1β capture beads (beads coated with antibodies against IL-1β) in appropriate buffer provided by the kit and were incubated at room temperature with rotation. Fluorescent detection antibodies were then added and the assay plate was incubated for an additional 2 hours. at room temperature, protected from light. Binding reactions were carried out at room temperature with rotation. This allows the fluorescent signal to be associated with the “capture antibody-IL-1β complex”, with the intensity of the fluorescence directly correlating to the quantity of bound IL-1β. A blank control (media only) and different dilutions of IL-1β standards (lyophilized standards were provided by the kit) were run in parallel to ensure the detection parameters for the unknown samples falls within the range of the standards. A plate shaker was used in each step to ensure thorough mixing. After the incubation steps were completed, plates were sampled using the HyperCyt™ high throughput flow cytometry platform.

The HyperCyt™ instrument consists of an autosampler (e.g. Gilson Gx-274), a peristaltic pump (Gilson Minipuls 3), tubing, and an inlet probe that connects to a compatible flow cytometer. These studies used an Accuri C6. The associated software includes HyperSip™ which controls the autosampler and is used to compose microtiter plate templates, and HyperView™ which is used to bin the time-resolved data files stored in flow cytometry standard 2.0 or 3.0 formats. The platform is set up as described (27). [REFERENCE Edwards B S, Young S M, Oprea T I et al. (2006) Biomolecular screening of formylpeptide receptor ligands with a sensitive, quantitative, high-throughput flow cytometry platform. [Nature Protocols 1: 59-66]

Results

Following Standard 3 Suture CCI, Mice Developed Profound Allodynia.

At baseline (BL), mice displayed no significant differences in hindpaw sensitivity (ipsilateral: F_{2,28}=0.782, p=0.468; contralateral: F_{2,28}=1.054, p=0.363). Previous studies have shown that the onset of allodynia for a standard CCI mouse model begins 3 days post-surgery with allodynia reaching full potential by day 10 post-surgery (Vanderwall et al. 2018). Following surgical manipulation, CCI mice displayed an increase in hindpaw sensitivity over a 10 day timecourse with a main effect of time (ipsilateral: F_{1.918},44.121=89.794, p=<0.001; contralateral: F_{1.632,37.539}=45.545, p=<0.001), surgery (ipsilateral: F_{1,23}=832.619, p=<0.001; contralateral: F_{1,23}=286.459, p=<0.001), and an interaction between time and surgery (ipsilateral: F_{1.918,44.121}=83.982, p=<0.001; contralateral: F_{1.632,37.539}=39.185, p=<0.001). Lastly, mice that received sham surgery remained at basal levels of hindpaw sensitivity throughout the experiment.

Mice that Received an Intravenous Injection of Pramipexole on Day 10 Post-Surgery at Doses of 5 μg or 0.5 μg Produced Complete Reversal from Allodynia within 2 Hours.

Behavioral analysis of hindpaw withdraw thresholds at hours 1, 2 & 3 hours post-injection revealed a main effect of surgery (ipsilateral: F_{1,23}=67.981, P=<0.001; contralateral: F_{1,23}=286.459, P=<0.001), a main effect of injection (ipsilateral: F_{2,23}=53.154, P=<0.001; contralateral: F_{2,23}=78.140, P=<0.001), an interaction between time and injection (ipsilateral: F_{3.916,45.040}=3.334, P=0.018) and an interaction between surgery and injection (ipsilateral: F_{2,23}=56.036, P=<0.001; contralateral: F_{2,23}=77.062, P=<0.001).

Mice that Received Pramipexole Returned to Allodynia by 24 Hours Post-Injection.

Behavioral hindpaw withdraw thresholds from hours 24-72 post-injection revealed a complete dissipation in the efficacy of intravenous Pramipexole at doses of 5 μg or 0.5 μg. Mice returned to allodynia by 24 hours post-injection and maintained allodynia throughout the 72 hour post-injection timecourse with a main effect of surgery (ipsilateral: F_{1,23}=727.240, P=<0.001; contralateral: F_{1,23}=418.259, P=<0.001).

Pramipexole Treatment Suppresses TLR4-Mediated Il-1□ Release in Activated Human Monocytes

Pramipexole suppresses the pro-nociceptive cytokine, interleukin-1beta (IL-1β), from being released after classic immune cell stimulation with the gram-negative bacteria cell wall particles, lipopolysaccharide (LPS).

Pramipexole (5 pg and 500 pg) does not significantly alter basal cellular levels of IL-1β. However, both 5 pg and 500 pg of pramipexole in the presence of LPS stimulation significantly blunts IL-1β release. These doses given to isolated cells in culture are 10,000 and 1000-fold lower than doses given for in vivo application for pain reversal, providing a rationale for testing these lower doses in future in vivo experiments to elucidate a dose-response function of pramipexole for pain reversal.

### REFERENCES

Bennett, G. J., and Y. K. Xie. 1988. ‘A peripheral mononeuropathy in rat that produces disorders of pain sensation like those seen in man’, Pain, 33: 87-107.
Bonin, R. P., C. Bories, and Y. De Koninck. 2014. ‘A simplified up-down method (SUDO) for measuring mechanical nociception in rodents using von Frey filaments’, Mol Pain, 10: 26.
Chaplan, S. R., F. W. Bach, J. W. Pogrel, J. M. Chung, and T. L. Yaksh. 1994. ‘Quantitative assessment of tactile allodynia in the rat paw’, J Neurosci Methods, 53: 55-63.
Dengler, E. C., J. Liu, A. Kerwin, S. Torres, C. M. Olcott, B. N. Bowman, L. Armijo, K. Gentry, J. Wilkerson, J. Wallace, X. Jiang, E. C. Carnes, C. J. Brinker, and E. D. Milligan. 2013. ‘Mesoporous silica-supported lipid bilayers (protocells) for DNA cargo delivery to the spinal cord’, J Control Release, 168: 209-24.
Liu, L., Y. Yin, F. Li, C. Malhotra, and J. Cheng. 2017. ‘Flow cytometry analysis of inflammatory cells isolated from the sciatic nerve and DRG after chronic constriction injury in mice’, J Neurosci Methods, 284: 47-56.
Martucci, C., A. E. Trovato, B. Costa, E. Borsani, S. Franchi, V. Magnaghi, A. E. Panerai, L. F. Rodella, A. E. Valseechi, P. Sacerdote, and M. Colleoni. 2008. ‘The purinergic antagonist PPADS reduces pain related behaviours and interleukin-1 beta, interleukin-6, iNOS and nNOS overproduction in central and peripheral nervous system after peripheral neuropathy in mice’, Pain, 137: 81-95.
Milligan, E. D., K. K. Mehmert, J. L. Hinde, L. O. Harvey, D. Martin, K. J. Tracey, S. F. Maier, and L. R. Watkins. 2000. ‘Thermal hyperalgesia and mechanical allodynia produced by intrathecal administration of the human immunodeficiency virus-1 (HIV-1) envelope glycoprotein, gp120’, Brain Res, 861: 105-16.
Noon, S., J. J. Sanchez, A. G. Vanderwall, M. S. Sun, J. R. Maxwell, S. Davies, L. L. Jantzie, T. R. Petersen, D. D. Savage, and E. D. Milligan. 2017. ‘Prenatal alcohol exposure potentiates chronic neuropathic pain, spinal glial and immune cell activation and alters sciatic nerve and DRG cytokine levels’, Brain Behav Immun, 61: 80-95.
Sanchez, J. J., S. Noor, S. Davies, D. Savage, and E. D. Milligan. 2017. ‘Prenatal alcohol exposure is a risk factor for adult neuropathic pain via aberrant neuroimmune function’, J Neuroinflannation, 14: 254.
Sommer, C., and M. Schafers. 1998. ‘Painful mononeuropathy in C57BL/Wld mice with delayed wallerian degeneration: differential effects of cytokine production and nerve regeneration on thermal and mechanical hypersensitivity’, Brain Res, 784: 154-62.
Vanderwall, A. G., S. Noor, M. S. Sun, J. E. Sanchez, X. O. Yang, L. L. Jantzie, N. Mellios, and E. D. Milligan. 2018. ‘Effects of spinal non-viral interleukin-10 gene therapy formulated with d-mannose in neuropathic interleukin-10 deficient mice: Behavioral characterization, mRNA and protein analysis in pain relevant tissues’, Brain Behav Immun, 69: 91-112.

1. A method of treating pain in a subject comprising administering to said subject a composition consisting essentially of an effective amount of pramipexole optionally in combination with a pharmaceutically acceptable carrier, additive or excipient, wherein said pramipexole is non-addictive.

2. The method according to claim 1 wherein said pramipexole is coadministered with an effective amount of at least one opioid analgesic agent.

3. The method according to claim 1 wherein pramipexole is the sole analgesic agent in the composition.

4. The method according to claim 2 wherein said opioid analgesic agent is selected from the group consisting of codeine, fentanyl, hydrocodone, hydromorphone, morphine, meperidine, oxycodone (atone or in combination with naloxone), buprenorphine, opium, tramadol, levorphanol, nalbuphine, tapentadol, butorphanol, propoxyphene, pentazocine, alfentanil, sufentanil, remifentanil and mixtures thereof.

5. The method according to claim 2 wherein said opioid analgesic agent is selected from the group consisting of codeine, fentanyl, hydrocodone, hydromorphone, morphine, meperidine, oxycodone (alone or in combination with naloxone) or a mixture thereof.

6. The method according to claim 1 wherein said pramipexole is coadministered with an effective amount of at least one non-opioid analgesic agent.

7. The method according to claim 6 wherein said non-opioid analgesic agent is selected from the group consisting of gabapentin, pregabalin, cannabidiol oil (CBD oil), nitric oxide and mixtures thereof.

8. The method according to claim 1 wherein said pain is acute pain.

9. The method according to claim 1 wherein said pain is chronic pain.

10. The method according to claim 1 wherein said pain is nociceptive pain.

11. The method according to claim 1 wherein said chronic pain is neuropathic pain.

12. The method according to claim 11 wherein said neuropathic pain is peripheral neuropathic pain.

13. The method according to claim 11 wherein said neuropathic pain is central neuropathic pain.

14. The method according to claim 1 wherein said pain is allodynia.

15. A method of reducing tolerance to an opioid analgesic agent administered to a patient or subject comprising coadministering to said subject an effective amount of premipexole to said patient or subject in combination with said opioid analgesic agent.

16. The method according to claim 15 wherein said opioid analgesic agent is selected from the group consisting of codeine, fentanyl, hydrocodone, hydromorphone, morphine, meperidine, oxycodone (alone or in combination with naloxone), buprenorphine, opium, tramadol, levorphanol, nalbuphine, tapentadol, butorphanol, propoxyphene, pentazocine, alfentanil, sufentanil, remifentail and mixtures thereof.

17. The method according to claim 15 wherein said opioid analgesic agent is selected from the group consisting of codeine, fentanyl, hydrocodone, hydromorphone, morphine, meperidine, oxycodone (alone or in combination with naloxone) or a mixture thereof.

18. The method according to claim 1 wherein said pramipexole is pramipexole dihydrochloride salt.

19. The method according to claim 2 wherein pramiprexole is administered at a daily dosage of about 0.05 mg. to about 5 mg. and said opioid analgesic agent is administered at a daily dosage of about 0.01 mg. to about 1 mg.

20. A method of reducing the likelihood of withdrawal from an opioid analgesic agent administered to a patient or subject comprising coadministering to said subject an effective amount of premipexole to said patient or subject in combination with said opioid analgesic agent.

21-30. (canceled)
